(19) United States
(12) Patent Application Publication
(10) Pub. No.: US 2023/0233749 A1
(43) Pub. Date: Jul. 27, 2023

(54) Chest Drainage System Securing Apparatus

(71) Applicant: Securisyn Medical, LLC, Littleton, CO (US)

(72) Inventors: Mark Bruning, Monument, CO (US); Greg Letendre, USAFA, CO (US); Arthur Kanowitz, Littleton, CO (US); Pierre Noel, Scottsdale, AZ (US); Kelly McKay, Southern Pines, NC (US); Patrick Parkinson, Denver, CO (US); Michael Brown, Thornton, CO (US); Dmitri Kyle, Denver, CO (US)

(21) Appl. No.: 18/011,223

(22) PCT Filed: Jun. 29, 2021
(86) PCT No.: PCT/US21/39729

§ 371 Date: Dec. 19, 2022

Related U.S. Application Data
(60) Provisional application No. 63/046,542, filed on Jun. 30, 2020.

(51) Int. Cl. A61M 1/00; A61M 25/00

(52) U.S. Cl. CPC A61M 1/84; A61M 25/0075; A61M 2025/0078; A61M 2025/0273

(57) ABSTRACT

A chest drainage system securing apparatus for securing and maintaining chest drainage systems in position following placement into a human or animal patient and preventing movement or unplanned removal thereof in response to the application of significant forces in any direction thereto, be they longitudinal, torsional/rotational or bending. The securement system includes retention devices secured to a chest tube which interact with an adhesive pad releasably attached to a patient to ensure proper positioning of the chest tube. The adhesive pad includes one or more Heimlich-type valves for sealing sucking chest wounds and for sealing an incision formed in a patients skin and chest for the placement and insertion of a chest tube.

### CROSS-REFERENCE TO RELATED APPLICATIONS

This application claims priority to U.S. Provisional Patent Application Ser. No. 63/046,542 filed on Jun. 30, 2020, the entire contents of which are incorporated herein by reference.

### RIGHTS OF THE U.S. GOVERNMENT

This invention was made with Government support under Contract No. FA8629-20-C-5028, The Government has certain rights in the invention.

### FIELD OF THE INVENTION

The present invention relates generally to human and veterinary medical devices. Specifically, the present invention relates to a drainage system and securing apparatus therefor adapted to maintain a tubular medical device in position following its insertion in a patient. More specifically, the present invention relates to a novel chest drainage system that combines two components that are typically supplied separately, namely a chest seal and a securing apparatus adapted to maintain the drainage system in position in a patient's pleural space for fluid removal therefrom. The chest seal of the present it is vented in one direction from an incision in a patient's chest in which an end of the drainage system is placed to a peripheral edge of the chest seal. The securing apparatus further prevents unintentional movement and/or removal of the drainage system from a patient or animal, particularly in emergency and/or field situations or in any situation where a force may be applied inadvertently thereto.

### BACKGROUND OF THE INVENTION

Catheters are used to drain fluids from the body; for example, chest tubes for draining air or fluid from the area, between a patient's chest wall and his or her lungs. A chest tube must be positioned accurately and maintained in the correct position in a patient, Subsequent movement of the patient or an inadvertently applied external force to a correctly positioned chest tube can lead to unintentional and potentially traumatic movement or even removal of a properly placed device completely. Localized tissue damage, hematoma, fluid accumulation and/or internal pressure caused by such movement or removal may result in severe injury and/or death. Moreover, a patient may restlessly move about and may consciously or subconsciously attempt to forcibly remove the drainage system, particularly if the patient is uncomfortable, experiencing breathing difficulty, anxiety, or panic. In the case of an animal patient, agitation may be particularly pronounced due to the animal's lack of cognitive awareness or understanding of its circumstances and an instinctual survival fight or flight response. A large animal or a carnivore can pose a serious danger not only to itself but also to a treating veterinarian and anyone in close proximity under such circumstances.

Chest tubes are elongate, semi-flexible tubes or catheters that a treating physician or a surgeon may insert in the area between a patient's chest wall and his or her kings, as noted above, an area known as the pleural space. Chest tubes are used to address a number of emergency and post-operative conditions such as a collapsed lung, a buildup of excess blood or other fluid in the chest cavity or to treat an infection. Chest tubes have been widely recognized as life-saving devices in field medical scenarios where military personnel may experience a collapsed lung as the result of a chest wound.

Medical emergencies may occur anywhere. Accordingly, emergency medical service personal (i.e., paramedics, Military Special Operations Medics), emergency department physicians, anesthesiologists, critical care clinicians, and surgeons may be called upon to insert chest drainage systems in out-of-hospital emergency settings as well as in field hospital and acute care hospital settings. Unintentional movement of a chest tube or a catheter of any type is not uncommon, particularly when the patient is in the process of being moved. This may occur during movement for example, from the field to an ambulance or helicopter, from an ambulance or helicopter to a hospital, also from one hospital to another hospital, from one area of the hospital to another area in the same hospital (imaging, laboratory, operating theater), or from a hospital to an outpatient rehabilitation facility. Anytime a patient is “moved”, even if it is a simple movement during a procedure in the hospital bed, unintentional movement of a catheter is a risk. Even repositioning a patient in a hospital bed, or in the case of an animal, in a recovery cage, may cause unintentional movement of their chest drainage tube.

U.S. Pat. No. 8,801.969 issued on Aug. 23, 2011, and U.S. Pat. No. 8,739,795 issued on Jun. 3, 2014, both to Arthur Kanowitz, the inventor of the present invention, disclose interlocking restraint or airway stabilization systems which address many of the problems set forth above associated with unplanned extubation of a patient that has been intubated with an endotracheal tube (ETT). Continuing research into ways of providing even more advanced and rapidly deployable interlocking restraint or airway stabilization systems has resulted in yet further improvements to the overall design of ETT system components. Significantly, this work has led to an expanded investigation of interlocking restraint systems adapted to cooperatively interact with catheters such as chest drainage devices in diverse applications to maintain the devices' correct positions in a patient and to prevent unintentional movement and/or removal thereof.

In view of the above, it will be apparent to those skilled in the art from this disclosure that a need exists for a chest drainage apparatus and, in particular, for an interactive securing and restraint system therefor adapted for field, emergency and post-procedure situations in hospitals that enables physicians, veterinarians and emergency medical response personnel to treat an impaired respiratory function under diverse and potentially unpredictable situations and circumstances. The novel chest drainage and securing apparatus of the instant invention not only advantageously protects a chest tube from occlusion and crushing, but also maintains it in its preferred position in a patient or animal while draining fluid or air from a patient's pleural space and prevents clinically significant movement thereof as a result of the application of multidirectional forces of significant magnitude to the device. The present invention addresses aforementioned needs in the art as well as other needs, all of which will become apparent to those skilled in the art from the accompanying disclosure.

### SUMMARY OF THE INVENTION

In accordance with the embodiments of the present invention, tubular drainage devices or catheters and interactive systems for securing them in position on a patient are structured and arranged to treat human or animal patients in veterinary applications) by draining fluids from specific anatomical areas or spaces located in a patient's body and, particularly, in a situation requiring the insertion of a chest tube, to facilitate its insertion into and to maintain its position in the anatomical area selected by the treating physician or emergency responder. The systems of the embodiments of the invention disclosed herein prevent clinically significant movement of tubular drainage devices such as chest drainage systems or chest tubes and the like in response to the application of forces in any direction thereto, namely, longitudinal, torsional/rotational or bending forces.

Unlike conventional prior art chest drainage systems, the chest drainage system herein disclosed includes respectively a chest tube for draining fluids from human or animal patients and a novel securing apparatus that interacts and cooperates with any chest tube to maintain the chest tube in position. The interactive components of the securing apparatus cooperate integrally with and engage the smooth outer surface of a standard chest tube to provide unparalleled strength and stability against movement in universal chest tube restraint applications, even when a tube becomes slippery from fluids and/or secretions, without applying any constricting pressure whatsoever to the tubular elements.

In an embodiment, a chest tube securing and restraint system is adapted to secure a chest tube in position in a patient, the chest tube having a semi-rigid elongate body which includes a patient end, a curvilinear portion operatively connected to the patient end, the curvilinear portion being adapted to permit insertion of the patient end of the chest tube through an external incision or wound aperture formed in an outer surface of the patient's skin and chest wall into the patient's chest cavity or pleural space, and a machine end. The chest tube further includes a continuous sidewall extending between the machine end and the curvilinear portion, thereby forming a hollow drainage conduit portion adapted to conform and be secured to the outer surface of the patient's chest. Air, blood, and other fluids are drawn out of the selected anatomical space in response to negative pressure supplied by a drainage or suction apparatus attached to the machine end of the chest tube.

In another embodiment, the continuous sidewall of the chest tube includes a smooth outer surface.

In an embodiment, a chest tube securing and restraint system includes a removable incision seal adhesive pad adapted to be placed on an outer surface of a patient's chest, the adhesive pad including an aperture formed therein and adapted to be positioned over an external incision or wound in a patient's skin and chest wall, a chest tube securing and sealing apparatus positioned over the aperture and operatively connected to the adhesive pad, the securing and sealing apparatus being adapted to position and secure a patient end of a chest tube in a patient's chest or pleural cavity in sealing engagement with the incision seal adhesive pad, and a tube clamp apparatus operatively connected to the adhesive pad and adapted to secure a machine end of a chest tube to an outer surface of a patient's chest.

In another embodiment, a chest tube securing and sealing apparatus includes a removable incision base frame secured to the adhesive pad and extending circumferentially around the aperture formed therein and positioned over an external incision or wound in a patient's chest, a removable elastic incision seal membrane positioned on the base frame, and a removable incision seal cap positioned on the elastic incision seal membrane and operatively connected to the base frame, whereby the elastic incision seal membrane is releasably secured to the adhesive pad in alignment with the aperture formed therein and positioned over the incision or wound in a patient's chest.

In still another embodiment, the removable elastic incision seal membrane includes a conically shaped body having a circular curvilinear side portion terminating in an upper flat end, the upper flat end having an aperture formed therein adapted to removably receive and form a seal about a patient end of individual chest tubes of different sizes.

In still another embodiment, the removable incision seal cap includes circular body portion extending circumferentially around a central axis and having an upper and a lower surface, inner and outer surfaces extending intermediate the upper and lower surfaces, and a plurality of angularly spaced apart, radially outwardly extending semi-circular recesses formed in the upper surface thereof, each of the radially outwardly extending recesses being structured and arranged to releasably receive a semi-rigid elongate body of a chest tube, whereby the chest tube is directionally aligned with the incision and the hollow drainage conduit portion is positioned in conforming alignment with the outer surface of the patient's chest during the drainage procedure.

In another embodiment, a guiding cover having a low-profile body portion structured and, arranged to reduce potential catch points on the chest tube and the securing and restrain system is releasably attached to the removable incision seal cap.

In still another embodiment, the low-profile guiding cover includes securement features on an upper surface thereof, the securement features being adapted to prevent movement of the chest tube following placement thereof.

In yet another embodiment, an interlocking securement and restraint system includes a tube clamp assembly secured to a portion of the removable incision seal adhesive pad, the tube clamp apparatus having a tube clamp base member or base, a tube clamp housing operatively connected to the base, a tube clamp top member operatively connected to the housing, and an adhesive pad attached to the base and which is adapted to conform and be secured to the outer surface of the patient's chest. The tube clamp top member includes a flexible beam operatively connected thereto and having an end portion adapted to releasably; engage and secure a chest tube, the end portion being structured and arranged to securely grip the chest tube without crushing the hollow drainage conduit portion thereof, whereby clinically significant movement of the chest tube with respect to a patient is prevented after it is inserted in a patient.

In another embodiment, the removable incision seal adhesive pad includes first and second separable segments operatively connected to one another along a perforated connection, the segments being adapted to be separated from one another and positioned at preselected locations on a patients outer chest and torso in response to the anatomical size of the patient.

In an embodiment, the first separable removable incision seal adhesive pad segment includes a chest tube securing and sealing apparatus operatively connected to the adhesive pad and positioned over an aperture formed therein extending circumferentially around and positioned over an external incision or wound in a patient's chest, and the second separable removable incision seal adhesive pad segment includes a tube clamp assembly secured thereto and adapted to releasably secure the semi-rigid elongate body of a chest tube, the second separable removable incision seal adhesive pad segment and the tube clamp assembly being adapted to be separated from the first separable removable incision seal adhesive pad segment and to be positioned on and releasably secured to a patient's outer chest and torso at a preselected spaced apart distance from the first separable removable incision seal adhesive pad segment in response to the anatomical size of the patient.

In still another embodiment, a chest drainage system includes a Heimlich-type one-way valve operatively connected to a machine end of a chest tube.

In yet another embodiment the Heimlich-type one-way valve is operatively connected to the machine end of the chest tube via a Luer lock connector.

In another embodiment, a securing apparatus may be installed on and/or removed from an aftermarket chest tube positioned previously in a patient without interrupting the function or operation of the chest tube.

These and other features, aspects and advantages of the present invention will become apparent to those skilled, in the art from the following detailed description of preferred embodiments taken in connection with the accompanying drawings, which are summarized briefly below.

### BRIEF DESCRIPTION OF THE DRAWINGS

Referring now to the attached drawings which form a part of this original disclosure:

FIG. 1A is a side perspective view of a chest drainage system installed on a patient in accordance with an embodiment of the present invention;

FIG. 1B is a cross sectional elevation view along section 1B-1B of a chest tube portion of the chest drainage system illustrated in FIG. 1;

FIG. 2A is a perspective view of a chest drainage system securing apparatus in accordance with an embodiment of the present invention;

FIG. 2B is a top plan view of the chest drainage system securing apparatus shown in FIG. 2A;

FIG. 3 is an exploded perspective view of the elements of a chest drainage system securing apparatus in accordance with an embodiment of the present invention;

FIG. 4A is a perspective view of a removable incision seal cap member of the chest drainage system of FIGS. 1-3;

FIG. 4B is a top plan view of the removable incision seal cap illustrated in FIG. 4A;

FIG. 4C is a bottom plan view of the removable incision seal cap illustrated in FIGS. 4A and 4B;

FIG. 5A is a perspective view of a removable incision seal elastic membrane member of the chest drainage system of FIGS. 1-3;

FIG. 5B is a top plan view of the removable incision seal elastic membrane illustrated FIG. 5A;

FIG. 5C is a cross sectional elevation view along section 5C-5C of the removable incision seal elastic membrane illustrated in FIGS. 5A and 5B;

FIG. 5A is a perspective view of a removable incision seal cap clamp member of the chest drainage system of FIGS. 1-3;

FIG. 6B is a side elevation view of the removable incision seal cap clamp illustrated in FIG. 6A;

FIG. 6C, is a top plan view of the removable incision seal cap clamp illustrated in FIGS. 5A and 6B;

FIG. 7A is a perspective view of a removable incision seal base frame member of the chest drainage system of FIGS. 1-3;

FIG. 7B is a top plan view of the removable incision seal base frame illustrated in FIG. 7A;

FIG. 7C is a cross sectional elevation view along section 7C-7C of the removable incision seal base frame illustrated in FIGS. 7A and 7B;

FIG. 8 is a perspective view of a removable incision seal O-ring member of the chest drainage system of FIGS. 1-3;

FIG. 9A is a perspective view of a removable incision seal plug member of the chest drainage system of FIGS. 1-3;

FIG. 9B is a top plan view of the removable incision seal plug illustrated in FIG. 9A;

FIG. 9C is a side elevation view of the removable incision seal plug illustrated in FIGS. 9A and 9B;

FIG. 10A is a perspective view of removable incision seal plug cap of the chest drainage system of FIGS. 1-3;

FIG. 10B is a side elevation view of the removable incision seal plug cap illustrated in FIG. 10A;

FIG. 10C is a bottom plan view of the removable incision seal plug cap illustrated in FIGS. 10A and 10B;

FIG. 10D is a cross sectional elevation view along section 10D-10D of the removable incision seal plug cap illustrated in FIGS. 10A, 10B; and 10C;

FIG. 11 is a perspective view of a removable incision seal plug tab of the chest drainage system of FIGS. 1-3;

FIG. 12 is a perspective view of a removable incision seal cap tether member of the chest drainage system of FIGS. 1-3;

FIG. 13A is a perspective view of a tube clamp top or cap member of the chest drainage system of FIGS. 1-3;

FIG. 13B is a top plan view of the tube clamp top or cap illustrated in FIG. 13A;

FIG. 13C is a side elevation view of the tube clamp top or cap illustrated in FIGS. 13A and 13B;

FIG. 13D is rear elevation view of the tube clamp top or cap illustrated in FIGS. 13A, 13B and 13C;

FIG. 14A is a perspective view of a tube clamp base member of the chest drainage system of FIGS. 1-3;

FIG. 14B is aside elevation view of the tube clamp base member illustrated in FIG. 14A;

FIG. 14C is a bottom plan view of the tube clamp base member illustrated in FIGS. 14A and 14B;

FIG. 14D is a cross sectional elevation view along section 14D-14D of the tube clamp base member illustrated in FIGS. 14A, 14B, and 14C;

FIG. 15A is a perspective view of a tube clamp housing member of the chest drainage system of FIGS. 1-3;

FIG. 15B is a top plan view of the tube clamp housing member illustrated in FIG. 15A;

FIG. 15C is a cross sectional elevation view along section 15C-15C of the tube clamp housing member illustrated in FIGS. 15A and 15B;

FIG. 16A is a top plan view of a removable incision seal adhesive pad member of the chest drainage system of FIGS. 1-3;

FIG. 16B is a top plan view of a portion of the removable incision seal adhesive pad illustrated in FIG. 16A enlarged to more clearly show the elements thereof;

FIG. 17A is a perspective view of a removable incision seal pouch member of the chest drainage system of FIGS. 1-3;

FIG. 17B is a top plan view of the removable incision seal pouch illustrated FIG. 17A;

FIG. 17C is a side elevation view of the removable incision seal pouch illustrated in FIGS. 17A and 17B;

FIG. 18 is a perspective view of a reduced profile securement cap or guiding cover in accordance with an embodiment of the present invention;

FIG. 19 is a side elevation view of the reduced profile securement cap or guiding cover illustrated in FIG. 18;

FIG. 20A, is a top plan view of a chest drainage system including a securement system and a Heimlich-type one-way valve in accordance with an embodiment; and

FIG. 20B, is a side plan view of the chest drainage system, securement system and Heimlich-type one-way valve illustrated FIG. 20A.

### DETAILED DESCRIPTION OF THE PREFERRED EMBODIMENTS

Selected embodiments of the present invention will now be explained with reference to the drawings. It will be apparent to those skilled in the art from this disclosure that the following descriptions of the embodiments of the present invention are provided for illustration only and not for the purpose of limiting the invention as defined by the appended claims and their equivalents.

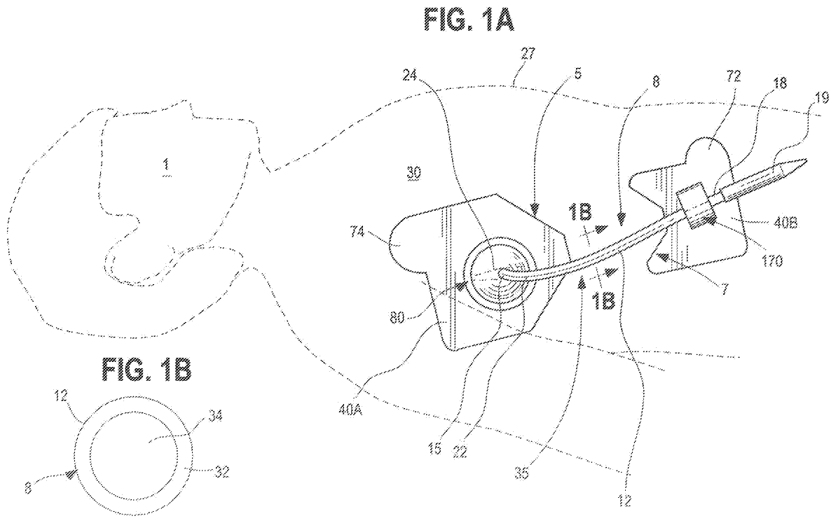

Referring initially to FIG. 1, a catheter drainage system for treating human (or animal patients in veterinary applications) by draining fluids from specific anatomical areas is shown installed on a patient shown generally at 1. By way of example and not of limitation, the catheter drainage system shown is a chest drainage system 5; however, it is to be understood that the system herein disclosed may be used with equal effectiveness with other types of catheter systems without departing from the scope of the present invention.

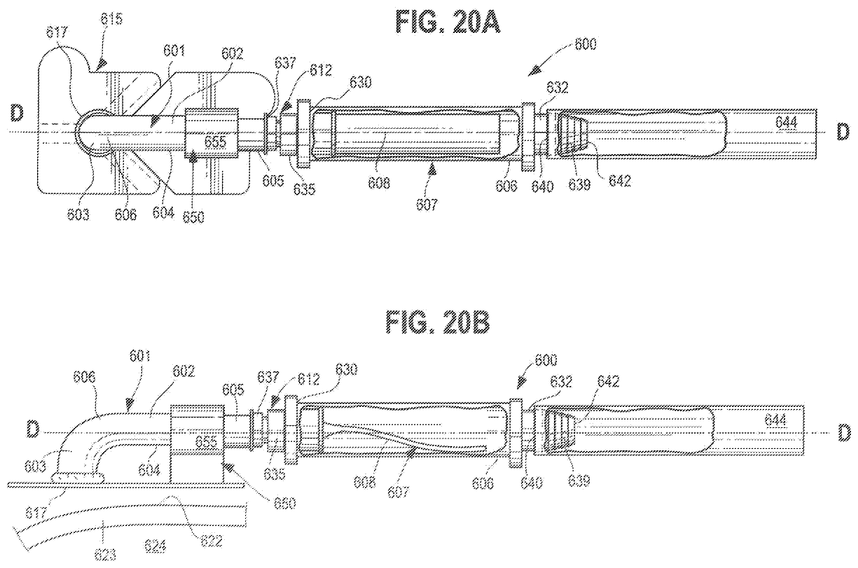

The chest drainage system 5 includes a chest drainage system securing apparatus or assembly 7, a chest tube 8 having a semi-rigid elongate body 12 which includes a patient end 15, a machine end 18, which in the embodiment shown includes protective cap 19, and a bend or curvilinear portion 22 (designated by the numeric identifier 606 in FIGS. 20A and 20B) that permits insertion of the patient end of the chest tube through an external incision 24 made in a patient's skin 27 and chest wall 30 into the patient's chest cavity or pleural space therebeneath. As best shown in FIG. 1A, the elongate body 12 of the chest tube 8 has a continuous sidewall 32 extends between the machine end and the curvilinear portion, thereby forming a hollow drainage conduit 34 adapted to conform and be secured to an outer surface 35 of a patient's chest 30. Air, blood, and other fluids are drawn out of the patient's chest cavity in response to negative pressure supplied by a drainage or suction apparatus attached to the machine end, as is known in the art.

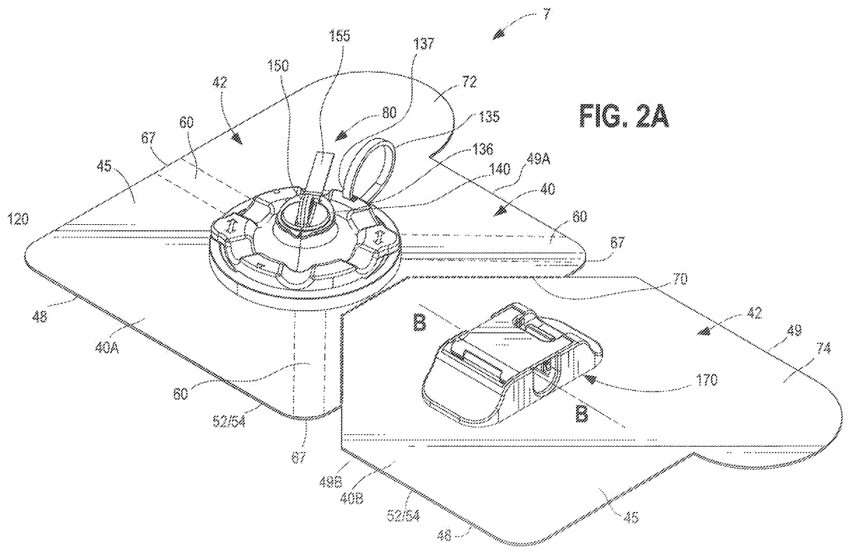

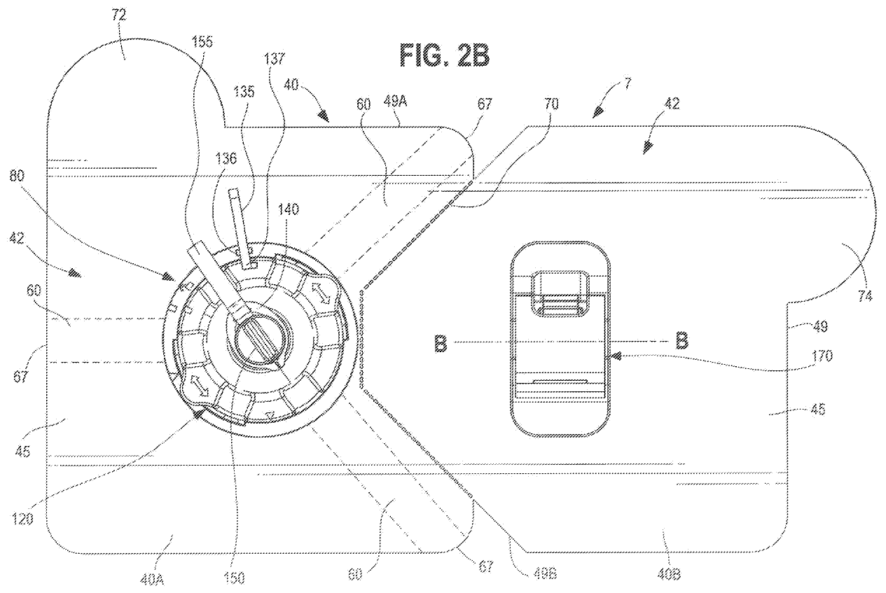

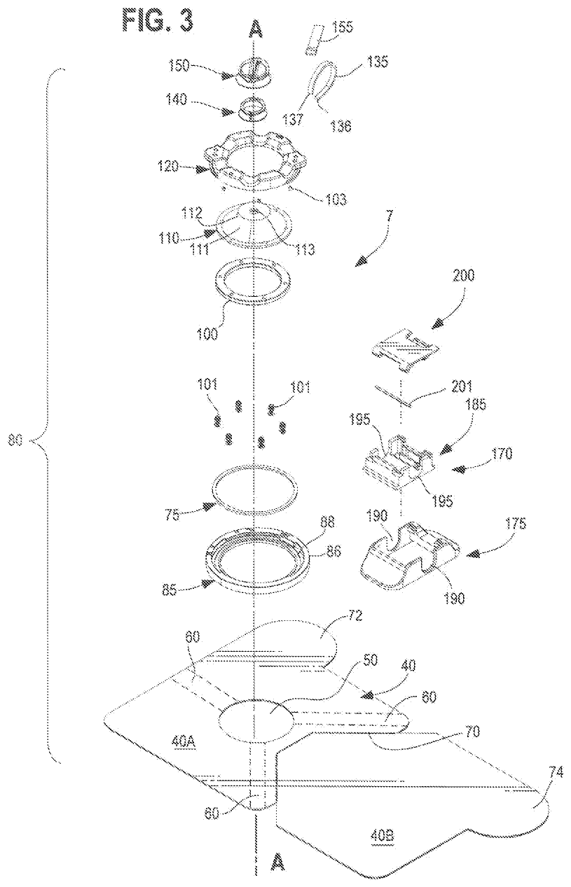

Referring to FIGS. 2A and 2B, and 3, the elements of the chest drainage system securing apparatus 7, also referred to herein as the securing apparatus for purposes of brevity, are depicted in greater detail. The securing apparatus includes a removable incision seal adhesive pad, or simply, adhesive pad 40, such as, by way of example and not of limitation, a HyFin® Vent Chest Seal offered by North American Rescue, LLC.

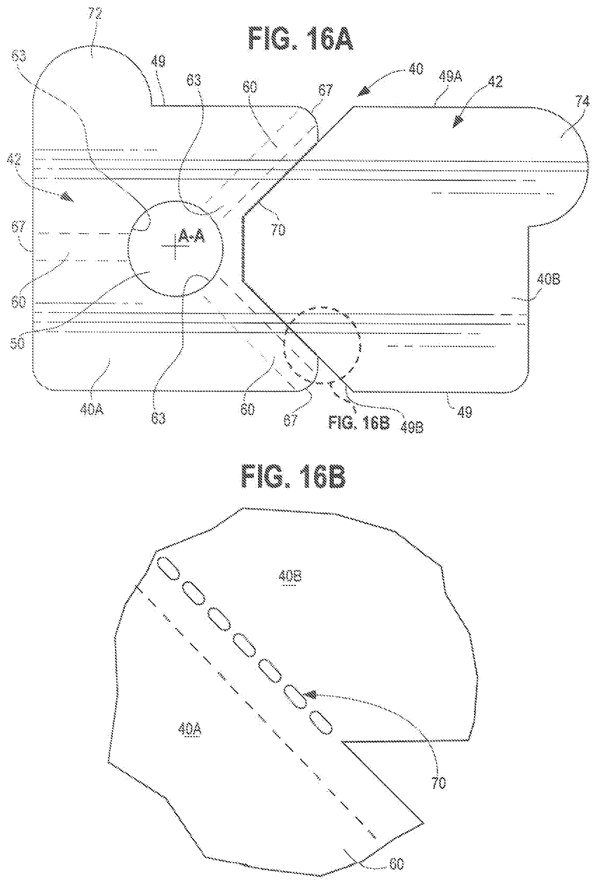

As best seen in FIGS. 16A and 16B, the removable incision seal adhesive pad 40 provides wound sealing capability, occlusive dressing properties, and Heimlich Valve-type one-way venting capabilities for treatment of various conditions and/or injuries such as open or sucking chest wounds, pneumothorax/tension pneumothorax caused by penetrating chest trauma such as may be caused by a stab or bullet wound. In the case of an open aperture in the chest cavity resulting from a penetrating chest trauma, the aperture must first be sealed to prevent airflow into the chest cavity during inhalation followed by creation of an incision for placement of a chest tube to remove blood and air from the pleural space. A seal is established via a silicone membrane body portion 42 of the adhesive pad 40, the silicone membrane body portion having an upper surface 45, a lower surface 48, a peripherally extending edge surface 49, a perforated seam or joint 70 extending intermediate first 49A and second 49B opposing portions of the peripherally extending edge surface, and an aperture 50 extending circumferentially about and coaxially along an axis A-A between the upper and lower surfaces. The aperture is adapted to be removably placed over the external incision 24 in a patient's skin 27 and chest wail 30, as described above. A hydrogel adhesive layer 52 covered by a release liner 54, which is removed prior to application of the incision seal adhesive pad 40 to a patient, extends across the entire lower surface 48 and is adapted to provide adhesive engagement with and conformation to a patient's outer chest and torso contour under adverse conditions, such as where the patient may be covered with blood, dirt, and other contaminants.

Referring back to FIG. 2B, the removable incision seal adhesive pad 40 has a plurality of internal vents or venting channels, each shown by dotted lines 60, embedded or formed in the silicone membrane body portion 42, which function in the same manner as a Heimlich or flutter valve, as will be described hereinbelow in greater detail with respect to the embodiments of FIGS. 20A and 20B, More specifically, following installation of the chest drainage system securing apparatus 7 on a patient and establishment of a seal, each of the plurality of vents is in fluid communication via a first end 63 thereof with the aperture 50 and a sealed region surrounding the external incision 24 and with the patient's chest cavity. Each of the plurality of venting channels is structured to allow air to escape from the patient's chest cavity via a second open end 67 thereof in fluid communication with the peripherally extending edge surface 4 during exhalation but to prevent air from flowing into the chest cavity during inhalation. The plurality of venting channels provide safety redundancy via the duplication of critical components or functions of the catheter drainage system, thus enhancing reliability of the system and patient survivability under adverse conditions.

Referring back to FIG. 16A, and more specifically to the details illustrated in FIG. 16B, the removable incision seal adhesive pad 40 is separable into segments 40A arid 40B, by breaking them apart along the perforated seam or joint 70 extending intermediate the two segments. While the chest drainage system securing apparatus 7 of the present invention may be provided in various sizes for patients having different anatomical geometries and configurations, depending upon the length of a chest tube 8 needed for the treatment of a patient, the adhesive pad may be broken along the perforated joint 70, thereby permitting placement of the segments 40A and 40B in a spaced apart arrangement on a patient's chest wall 30 to provide full support, securement and stability to the chest tube, as shown in FIG. 1. Each segment 40A and 40B has a tab 74 respectively, each tab adapted to facilitate handling and positioning of the adhesive pad segment to which it is attached on a patient.

In addition to the removable incision seal adhesive pad 40, the chest drainage system securing apparatus 7 has two separate subcomponents or subassemblies which interact and cooperate with the adhesive pad and with each other to provide the exceptional strength and stability of the securing apparatus of the present in The first of these subassemblies is an incision seal subassembly 80 which is operatively connected to adhesive pad segment 40A. The second subassembly is a tube clamp subassembly 170 which is operatively connected to adhesive pad segment 40B. The features and orientation of the subassemblies with respect to one another are shown in FIGS. 2A and 2B.

Referring to the exploded views of the subassemblies 80 and 170 presented in FIG. 3, the individual components of each are shown in greater detail. The incision seal subassembly 80 includes a removable incision seal base frame 85 which is structured and arranged to fit securely into and in sealing engagement with aperture 50 formed in adhesive pad segment 40A. The incision seal base frame has circular or ring-shaped body 80 extending circumferentially about axis A-A. The ring-shaped body includes an annular stepped upwardly extending surface 88 formed on an inner, circumferentially extending recessed face 89, the annular stepped surface being adapted to receive a removable incision seal O-ring 75 and a removable incision seal cap clamp 100 therein.

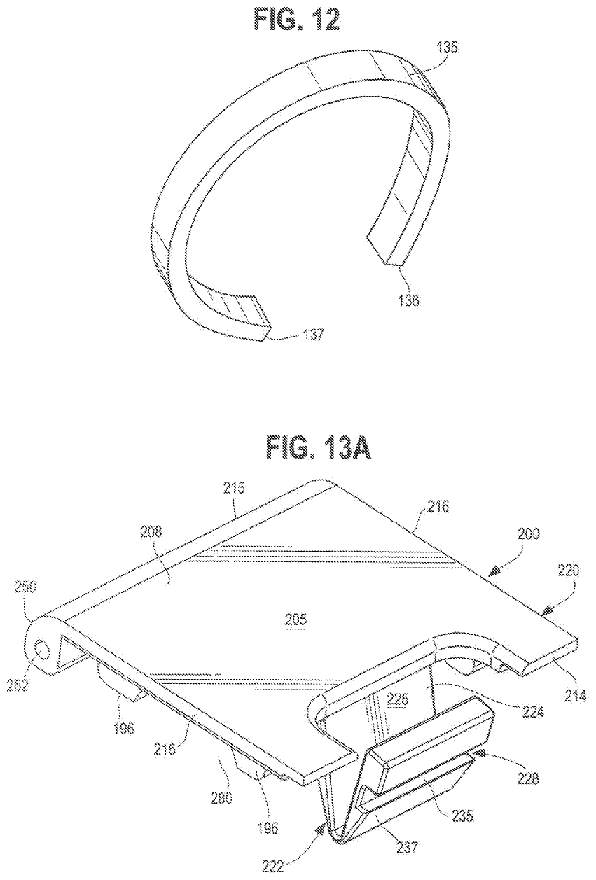

The removable incision seal cap clamp 100 is operatively connected to a removable incision seal elastic membrane 110 positioned intermediate the cap clamp 100 and a removable incision seal cap 120 via a plurality of removable cap screws 101 each cap screw being threadably received in a corresponding one of a plurality of cooperating cap inserts 103 positioned in the removable incision seal cap 120. As will be described in greater detail below, during the installation process, the incision seal cap 120 and the in seal elastic membrane 110 must be removed to allow access to the incision 24 in the patient's skin 27 and chest wall 30. Under adverse and potentially low light or totally dark conditions, potential loss of the incision seal cap 120 would render the chest drainage system securing apparatus 7 useless, thereby compromising the ability of attending personnel to provide life-saving treatment to the patient. Accordingly, to prevent its loss, tether 135 is attached at a first end 136 thereof to the removable incision seal base frame 35 and attached at a second end 137 thereof to the removable incision seal cap 120, as shown in FIGS. 2A and 2B. An enlarged perspective view of the tether is presented in FIG. 12 to more clearly illustrate the features thereof.

The incision seal subassembly further includes a removable incision seal plug or split ring lock 140 which is adapted to fit circumferentially around an upper neck or curved nipple shaped body portion 111 of the incision seal elastic membrane 110 and cooperates with an incision seal plug cap 150 removably inserted in an aperture 113 formed a flat upper end 112 of the nipple shaped body portion 111 of the incision seal elastic membrane 110 to isolate the area located intermediate the body portion 111 of the incision seal elastic membrane 110 and the patient's skin 27 and chest wall 30. A tab 155 is attached to the incision seal plug or split ring lock 140 which when grasped and pulled in a direction radially outwardly away from the axis A-A, facilitates removal of the seal plug or split ring lock from the subassembly 80.

The tube clamp subassembly 170 includes a tube clamp housing 175 operatively connected to adhesive pad segment 40B. A tube clamp bottom or base member 185 is releasably connected to the tube clamp housing. Both the tube clamp housing and base member have aligned, oppositely disposed and spaced apart apertures or slots 190, 195 respectively formed therein, each aperture being structured and arranged to releasably receive and secure a machine end 18 of a chest tube 8. A tube clamp top or cap member 200 is pivotably connected to the tube clamp base member 185 by a dowel pin 201 and is adapted to close in locking engagement with the tube clamp base member to secure a machine end 18 of a chest tube 8.

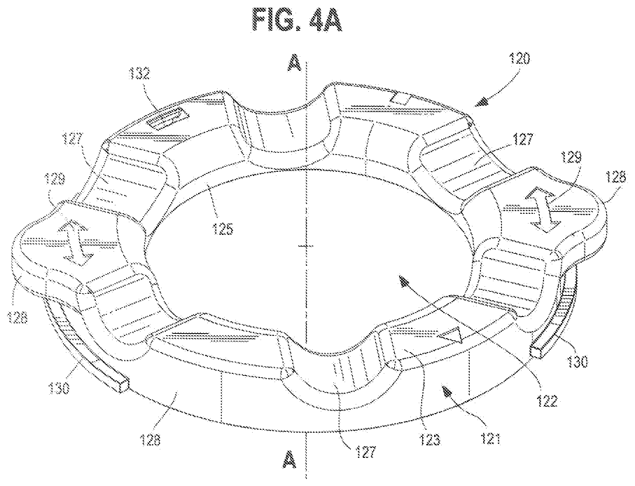

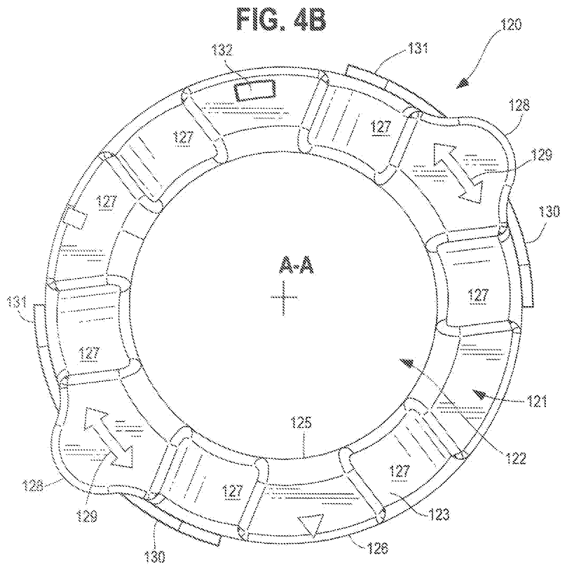

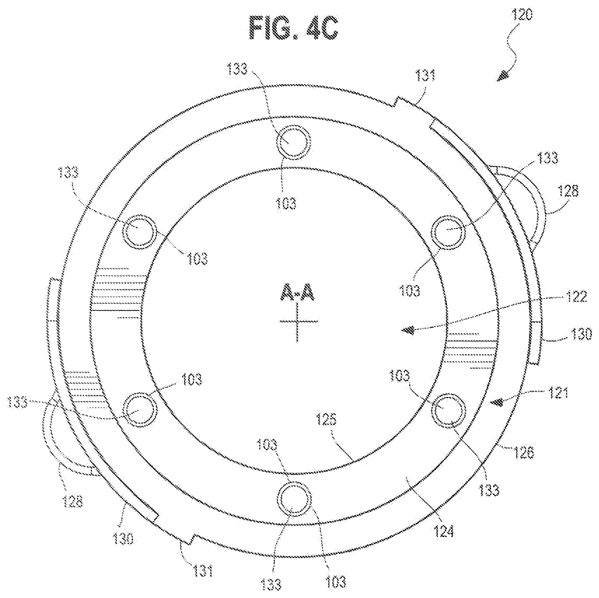

The elements of removable incision seal cap 120 are illustrated in greater detail in FIGS. 4A-4C, The incision seal cap includes a cylindrically or disk shaped body 121 extending circumferentially about axis A-A and having an aperture 122 formed therein and extending intermediate an upper surface 123 and a lower surface 124, and oppositely disposed parallel inner and outer surfaces 125 and 126 extending in a direction parallel to axis A-A. The upper surface 123 has a plurality of radially outwardly extending uniformly spaced apart semicircular channels 127 formed therein, each of the plurality of channels being adapted to selectively receive and align a patient end 15 of a chest tube 8 with an external incision 24 made in a patient's skin 27 and chest wall 30. The body 121 further includes a pair of oppositely disposed tabs 128 having directional arrows or locking indicators 129 formed thereon to indicate that the incision seal cap is aligned for installation and either locked or unlocked in a manner similar to the locking mechanism of a pressure cooker lid. Manual rotational manipulation of the incision seal cap via tabs 128 urges flanges 130 secured to or integrally formed with and extending radially outwardly from outer surface 126 into locking engagement with mating slots 93, 94 described below and formed in the incision seal base frame 85 until stop members 131 operatively engage an end 95, 96 of each of the mating slots, thus providing tactile feedback to treating personnel that the incision seal cap is locked. The alignment and simple twist locking and unlocking features provide not only strength to the chest drainage system securing apparatus, but also speed of application which is critical in emergency situations where minutes or even seconds can make a life-saving difference.

The body 121 of the removable incision seal cap 120 further includes a slot or aperture 132 adapted to receive the second end 137 of the tether 135 to secure the incision seal cap to the incision seal base frame 85. As shown in FIG. 4C, the body also has a plurality of equally spaced apart apertures 133 formed therein, each of the apertures being adapted to receive one of the plurality of cap inserts 103. Each of the plurality of cap inserts is adapted to threadably receive one of the plurality of cap screws 101 to secure the incision seal base clamp 100 and the incision seal elastic membrane 110 to the incision seal cap 120.

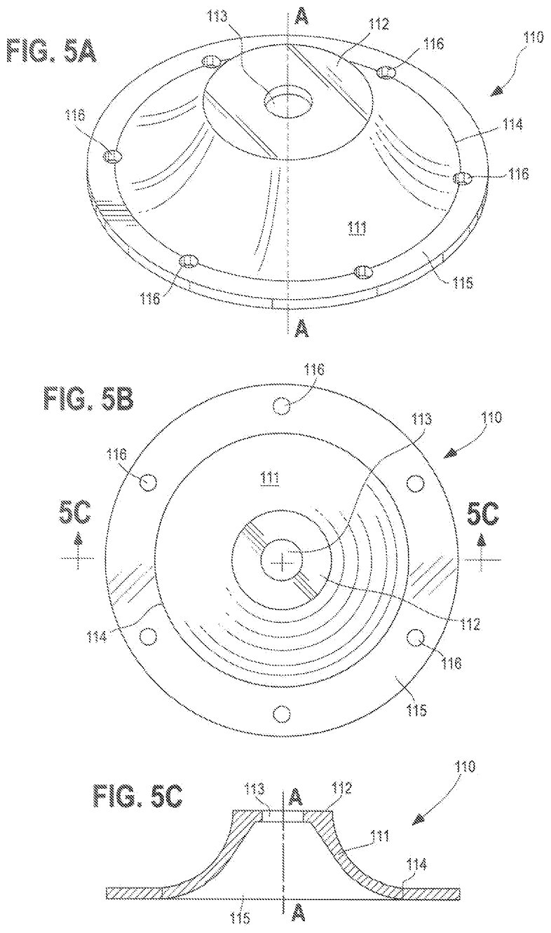

The details of the incision seal elastic membrane 110 are illustrated in FIGS. 5A-5C As noted above, the incision seal elastic membrane includes a conically or curved, nipple shaped membrane body 111 formed of silicone or a material possessing similar sealing properties. The membrane body has a flat upper end 112. An aperture 113 adapted to releasably receive a patient end 15 of a chest tube 8 in sealing engagement therein is formed in the flat upper end. The membrane further includes a lower end 114 operatively connected to an inelastic flange 115 having a plurality of apertures 116 formed therein, each aperture adapted to receive one of the plurality of cap screws 101, as hereinabove described,

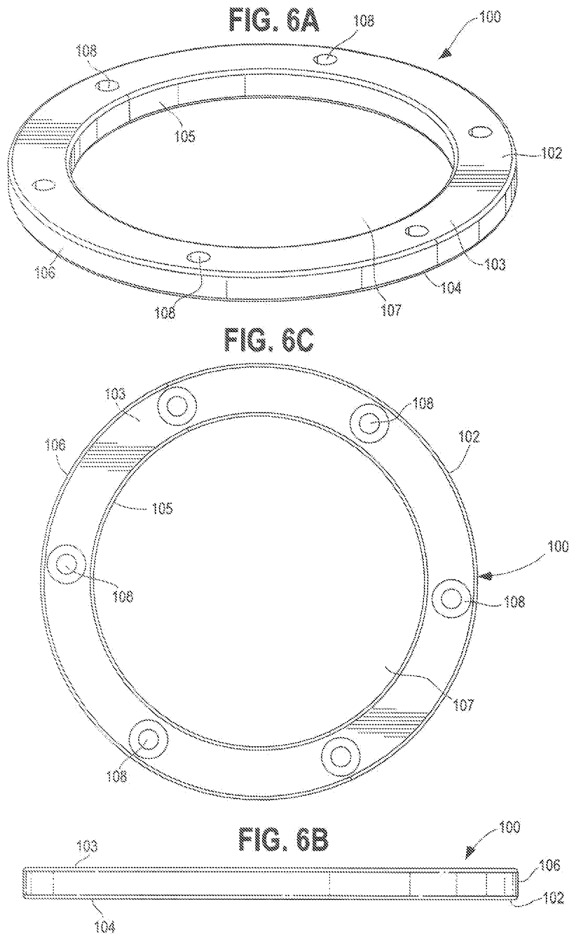

The structural details of the removable incision seal cap clamp 100 is depicted in FIGS. 6A-6C. The incision seal cap clamp includes a circular disk or washer shaped body 102 extending circumferentially about axis A-A, the body having flat parallel upper and lower surfaces 103, 104 and parallel inner and outer circular shaped surfaces 105, 106 extending perpendicularly therebetween and forming an aperture 107 extending through the body. The body 102 further include a plurality of countersunk apertures 108 extending therethrough, each aperture being adapted to receive one of the plurality of cap screws 101 which threadably secure the incision seal cap clamp, the incision seal elastic membrane 110, and the removable incision seal cap 120 to one another.

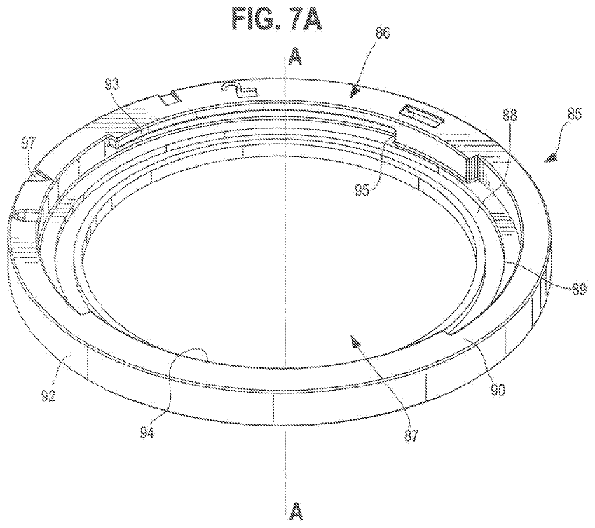

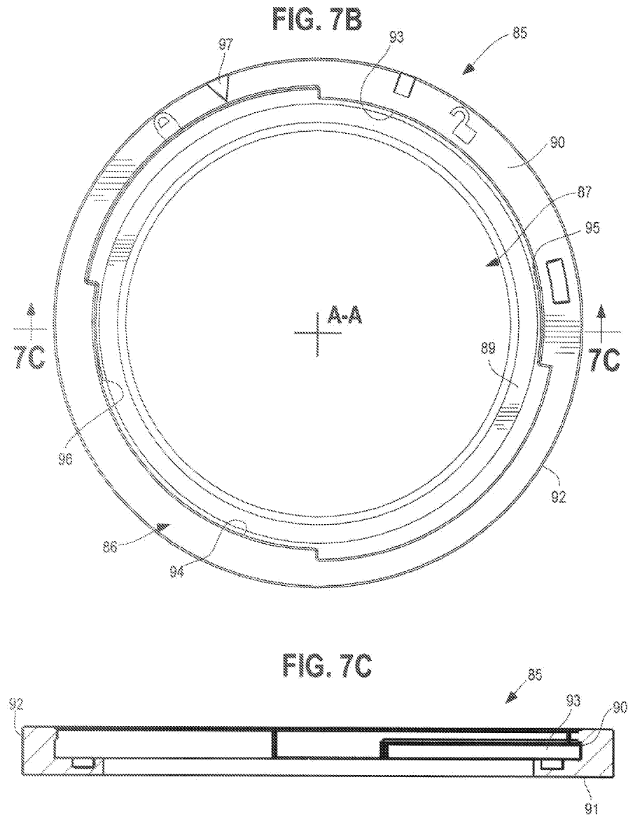

Referring now to FIGS. 7A-7C, the structural details of the removable incision seal base frame 85 are shown. The incision seal base frame has a circular or ring-shaped body 86 extending circumferentially about axis A-A forming an aperture 87. The ring-shaped body includes an annular stepped upwardly extending surface 88 formed on an inner, circumferentially extending recessed face 89, the annular stepped surface being adapted to receive a removable incision seal O-ring 75 and the removable incision seal cap clamp 100 therein. The incision seal base frame further includes flat parallel upper and lower surfaces 90, 91, an outer circular surface 92 extending intermediate the upper and lower surfaces in a direction parallel to axis A-A, and a pair of oppositely disposed circumferentially extending slots 93, 94 adapted to rotatably receive the flanges 130 operatively connected to the incision seal cap to lock the incision seal cap to the incision seal base frame. Each of the slots 93, 94 includes an end 95, 96 respectively structured and arranged to engage the incision seal ring stop members to provide tactile feedback to treating personnel that the incision seal cap is locked. The upper surface 90 further includes a lock/unlocked position indicator 97 and a bracket or hook member 98 adapted to receive the first end 136 of tether 135 to secure to secure the incision seal cap 120 to the incision seal base frame.

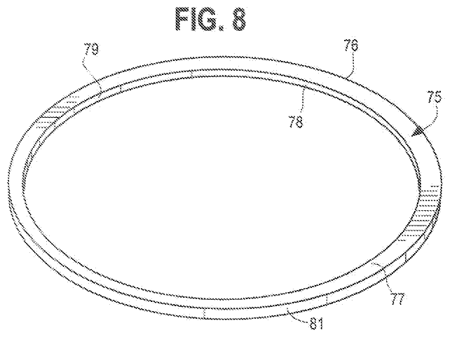

FIG. 8 illustrates the details of the removable incision seal O-ring 75. The O-g has a circular body member 76 having oppositely disposed, parallel flat upper and lower surfaces 77, 78 and inner and outer circumferential surfaces 79, 81. The incision seal O-ring is seated on the annular stepped upwardly extending surface 88 of the incision seal base frame intermediate the incision seal base frame and the incision seal cap clamp 100.

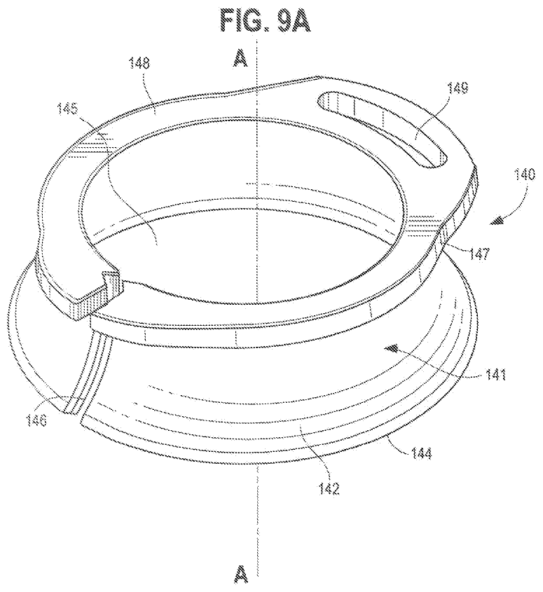

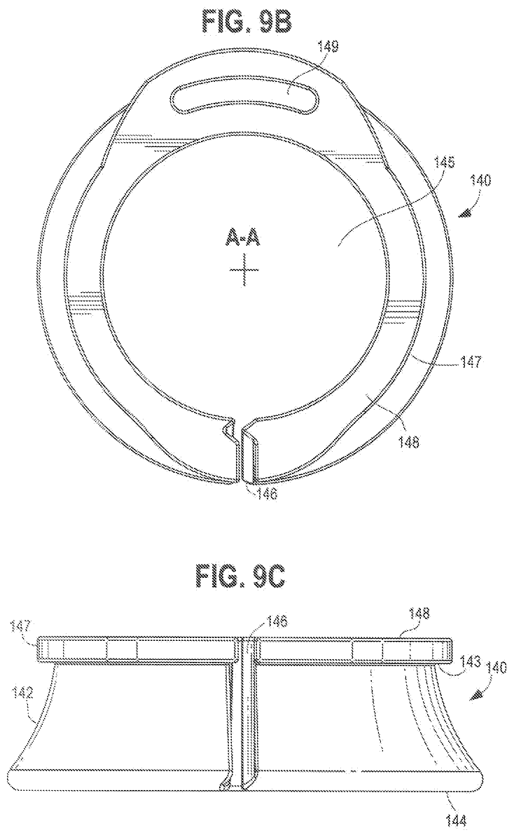

Referring to FIGS. 9A-90, the removable incision seal plug or split ring lock 140 is shown in greater detail. The incision seal plug includes a truncated conically shaped circular body 141 having a curved side 142 conforming to the cured side of the nipple shaped body portion 111 of the incision seal elastic membrane 110. The conically shaped body extends circumferentially around axis A-A intermediate upper and lower parallel edges 143, 144 and forms an aperture 145 extending therethrough. The curved side of the conically shaped circular body is split by a gap or opening 146 formed therein which extends the length thereof from the upper to the lower edge, thereby facilitating the split ring lock's insertion onto and removal from a chest tube. A flange 147 having a flat upper surface 148 is operatively connected to the upper edge 143. A slot 149 formed in flange is adapted to receive a loop 156 formed in and end 157 of the tab 155, the details of which are illustrated in FIG. 11.

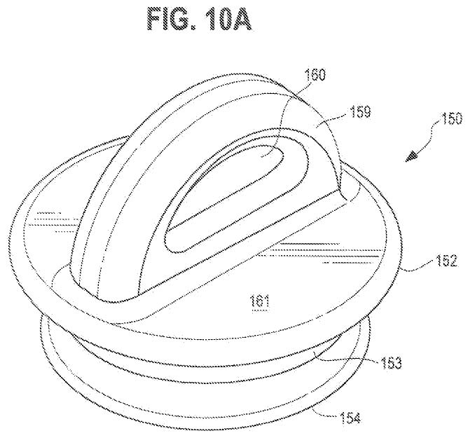

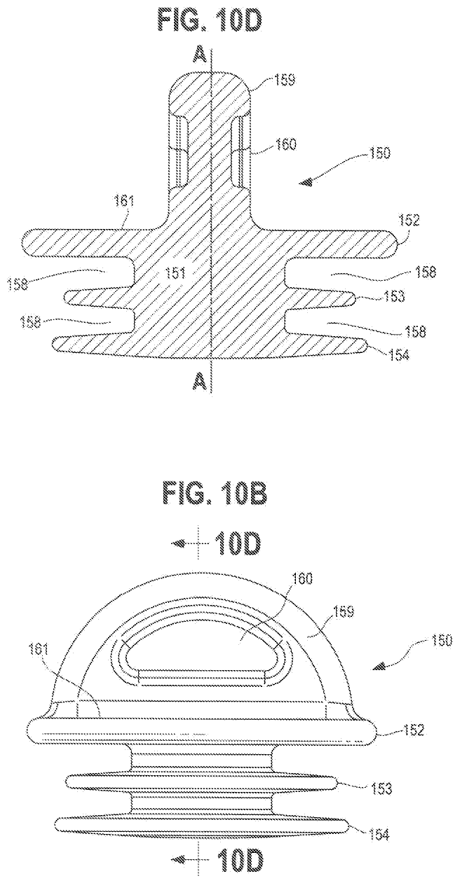

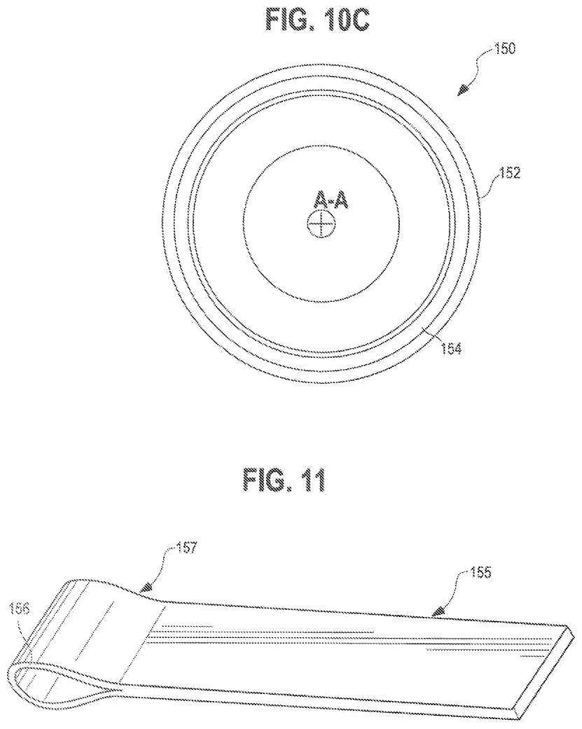

As discussed above, the removable incision seal plug or split ring lock 140 cooperates with the removable seal plug cap 150 removably inserted in aperture 113 formed a flat upper end 112 of the nipple shaped body portion 111 of the incision seal elastic membrane 110 to isolate the area located intermediate the body portion 111 of the incision seal elastic membrane 110 and the patient's skin 27 and chest wall 30. Among other possible configurations, the plug cap may be similar in configuration and function to a wine stopper, a threaded insert, or a sealing film secured over the flat upper end of the nipple shaped body portion that may be conveniently peeled off. By way of example and not of limitation, exemplary structural elements of a seal plug cap 150 are shown in FIGS. 10A-10D, the seal plug cap has a central cylindrically shaped body 151 from which three cylindrically shaped flanges 152, 153 and 154 operatively connected thereto or formed integrally therewith extend radially outwardly. Each of the flanges has a different diameter, the largest one being the topmost flange 152, and the smallest one being the middle-most flange 153. The flanges are evenly spaced apart and are separated by a recess 158 of the same width positioned intermediate two adjacent flanges, thereby establishing a tight seal when inserted correctly in the removable incision seal plug or split ring lock 140. A handle 159 having an aperture 160 formed therein is secured to an upper surface 161 of the flange 152 for ease of insert or removal of the incision seal plug or split ring lock 140.

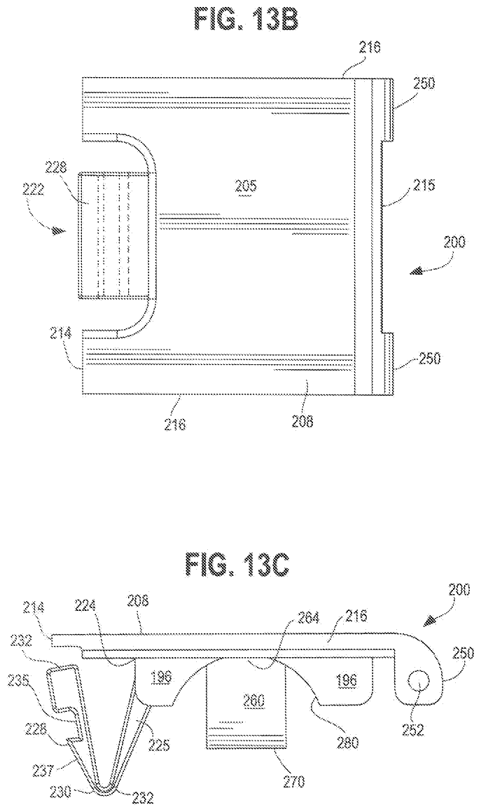

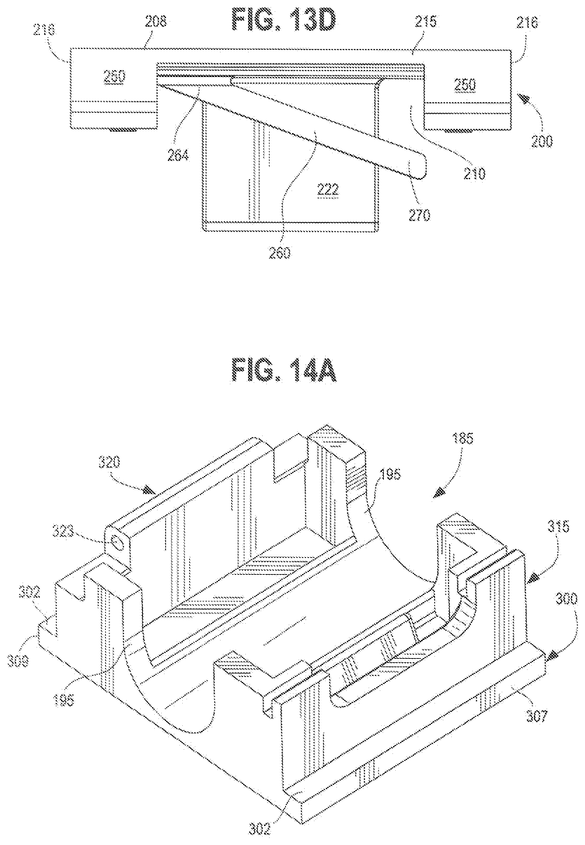

Referring next to FIGS. 13A-15C, the elements of the various components of the tube clamp subassembly 170 are described in detail. As shown in FIG. 13A the tube clamp subassembly includes a tube clamp top or cap member 200 which is pivotably connected to a tube clamp base member 185 by a dowel pin 201 (FIG. 3) and is adapted to close in locking engagement with the tube clamp base member to secure a machine end 18 of a chest tube 8. The tube cap member 200 includes a flat square-shaped body 205 having an upper surface 208, a lower surface 210, a pair of front and rear oppositely disposed edges 214, 215 and a pair of oppositely disposed side edges 216 extending intermediate the upper and lower surfaces, the edges defining a periphery 220 of the square shaped body.

A V-shaped resilient elastomeric latch member 222 is operatively connected to the lower surface 210 of the body 205 at a first wide end 224 of tapered leg member 225 thereof. A second tapered leg member 228 having a narrow end 230 and a wide end 232 is operatively connected via the narrow end 230 to a second narrow end 232 of the first tapered leg member 225. The elastomeric properties of the material bias the tapered leg members such that they are urged apart at the wide ends, thus urging a recess 235 formed on an outer surface 237 of the second tapered leg into releasable locking engagement with a mating looking mechanism on the tube clamp bottom or base member 185.

Referring to FIGS. 13C and 13D, the clamp top or cap member 200 further includes a pair of spaced apart ears or hinge members 250 operatively connected to the rear edge 215, each of the hinge members having an aperture 252 formed therein in axial alignment with one another and adapted to receive the dowel pin 201 which pivotally connects the top or cap member 200 to the tube clamp base member, as will be described in greater detail below. The clamp top member also has a flexible beam member 260 secured at a first end 264 to the lower surface 210 thereof. The flexible beam member is formed of an elastomeric material and is structured and arranged so that when the clamp top member is closed over the elongate body portion 12 or a machine end portion 8 of a chest tube and in locking engagement with the top clamp bottom or base member 185, a second end 270 of the flexible beam member is urged into locking engagement with whatever portion of a chest tube is positioned in the base member. Importantly, the flexible beam member is designed to apply significant retention forces to a chest tube; however, the force vectors have been engineered so as to not crush the chest tube. In an embodiment, the flexible beam, top clamp member, and tube clamp base member may have a rubberized or a silicone liner material coating on the components thereof which contact a chest tube to enhance frictional properties and corresponding retention strength of those components. The top clamp member also includes a pair of spaced apart, aligned apertures or slots 280 respectively formed in downwardly extending side members 196 attached to the bottom surface 214, each aperture being structured and arranged to releasably receive and secure a machine end 18 of a chest tube 8.

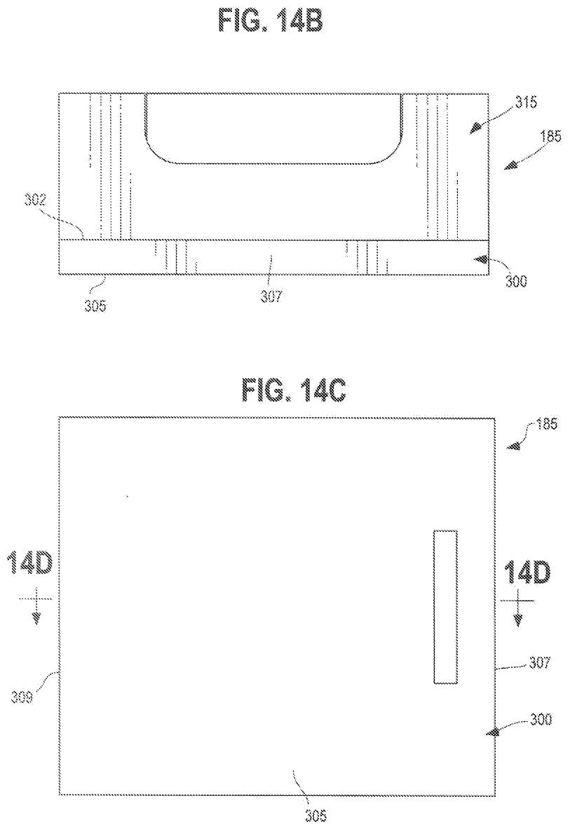

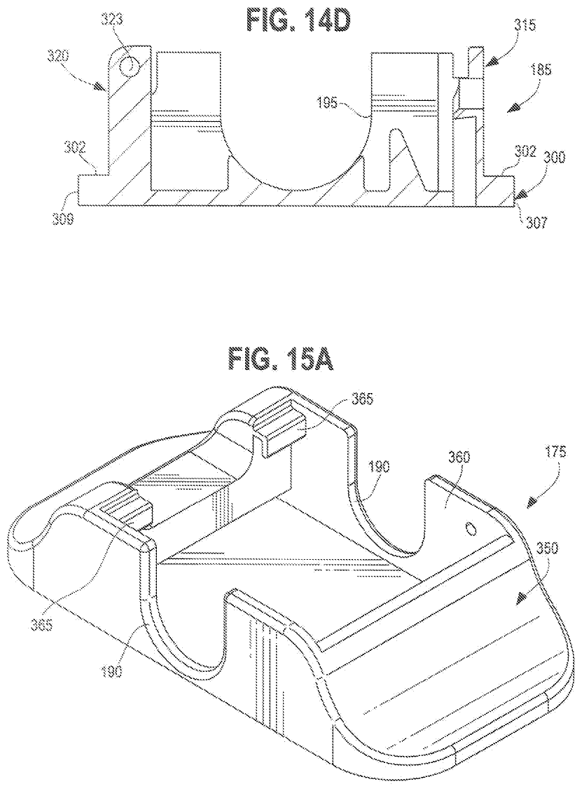

The features of the tube clamp base member 185 are shown in detail in FIGS. 14A-14D. The base member includes a base plate 300 having a top surface 302, a bottom surface 305, and front and back parallel edge surfaces 307, 309 extending intermediate the top and bottom surfaces. A front locking assembly 315 extends upwardly from the top surface 302, the front locking assembly being adapted to releasably interact locking engagement with the V-shaped resilient elastomeric latch member 222 of the tube clamp top or cap member 200. The front locking assembly and V-shaped resilient elastomeric latch member are structured and arranged so that when they engage, they emit an audible click indicating to a treatment provider or first responder that the machine end of the chest tube being installed has been captured by the tube clamp subassembly 170.

The tube clamp base member 185 further includes a clamp top or cap attachment member 320 extending upwardly from the top surface 302. The clamp top or cap attachment member includes an aperture 323 formed therein, the aperture being adapted to receive the dowel pin 201 extending through pair of spaced apart ears or hinge members 250, thereby pivotally connecting the top or cap member 200 to the tube clamp base member. When the top or cap member 200 is secured in locking engagement with the tube clamp base member 185, the spaced apart, aligned apertures or slots 280 respectively formed in downwardly extending side members 196 are aligned with upwardly extending spaced apart apertures or slots 195 and cooperate with one another to retain and secure the machine end of the chest tube positioned therein.

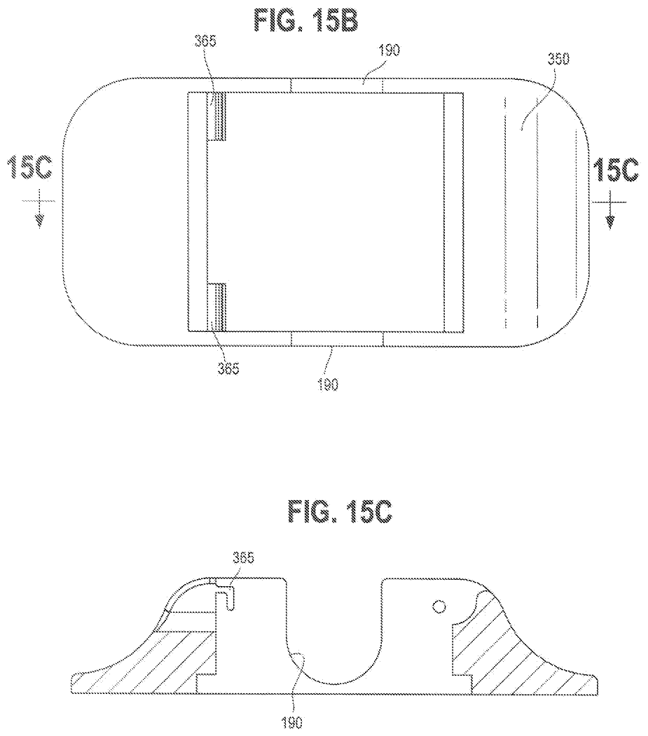

FIGS. 15A-15C illustrate the structure and operative features of tube clamp housing 175, The tube clamp housing includes a base member 350 adapted to be releasably secured to the segment 408 of removable incision seal adhesive pad 40. The base member includes a rectangularly shaped cavity 360 adapted to releasably receive and secure the tube clamp base member 185 therein, When the tube clamp base member 185 is inserted into the rectangularly shaped cavity, it is retained in place by a pair of L-shaped clips 365 operatively connected to or integrally formed with the, base member. The base member further includes upwardly extending spaced apart slots 190 formed in first and second side panels operatively secured to or formed integrally with the base member. The slots 190 are structured and arranged to align with the slots 195 formed in the tube clamp base member 185 and also with the spaced apart, aligned apertures or slots 280 respectively formed in downwardly extending side members 196 of the top or cap member 200, the respective slots cooperating with one another to retain and secure the machine end of the chest tube positioned therein.

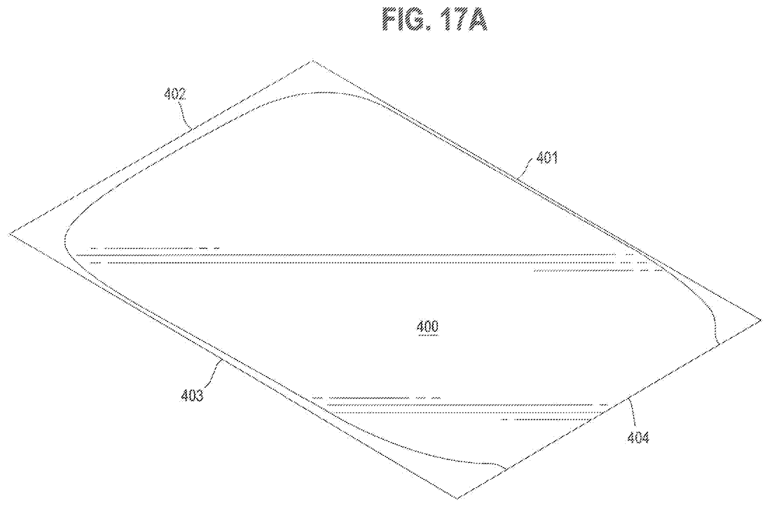

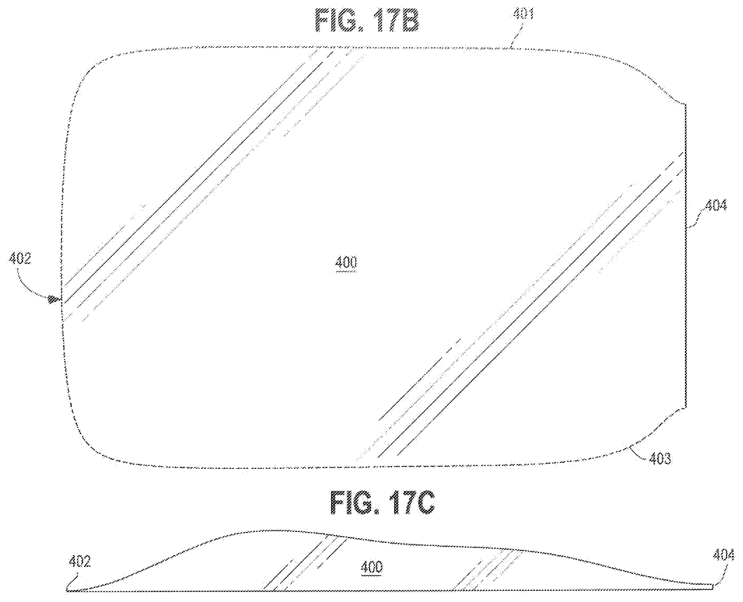

As noted earlier, among other applications, the chest tube securing and restraint system 7 of the present invention is designed and intended for the treatment and management of open chest wounds caused by penetrating trauma in hostile environments. Rephrased in the vernacular, the chest tube securing and restraint system of the present invention is designed for treatment of warfighters who have sustained penetrating chest trauma (sucking chest wounds) as a result of being hit in the chest by enemy gunfire or by bomb, grenade, or artillery fragments. Wound treatment under such conditions must be delivered quickly, calmly and competently with a high probability that the wounded individual's clothing and chest area will be covered with blood, dirt and other contaminants. Applying a dirty, contaminated chest tube securing and restraint system to a casualty under such conditions will only make things worse. Moreover, it may be more likely that cleaning of injured individual's skin surface may be easier to accomplish than cleaning a contaminated dressing. Accordingly, the chest tube securing and restraint system 7 of the present invention is provided to field personnel in a clean, sealed, compact package such as the pouch 400 illustrated in FIGS. 17A-17C, that not only keeps the system clean until it is needed, but also compresses it to occupy less volume. As a result, field medical treatment providers can efficiently pack more systems into a remote area or combat zone, thus providing the ability in the field to treat and manage more individuals suffering from chest wound injuries. The pouch 400 includes at least two layers of a waterproof material such as a waterproof plastic or aluminum foil cleaned and sealed along three sides, 401, 402, and 403 before placing one or more systems 7 therein. The pouch may or may not be evacuated prior to sealing a fourth edge 404; however, vacuum sealing thereof will reduce the overall volume of the packaged system and enable a greater number of systems to be carried into the field.

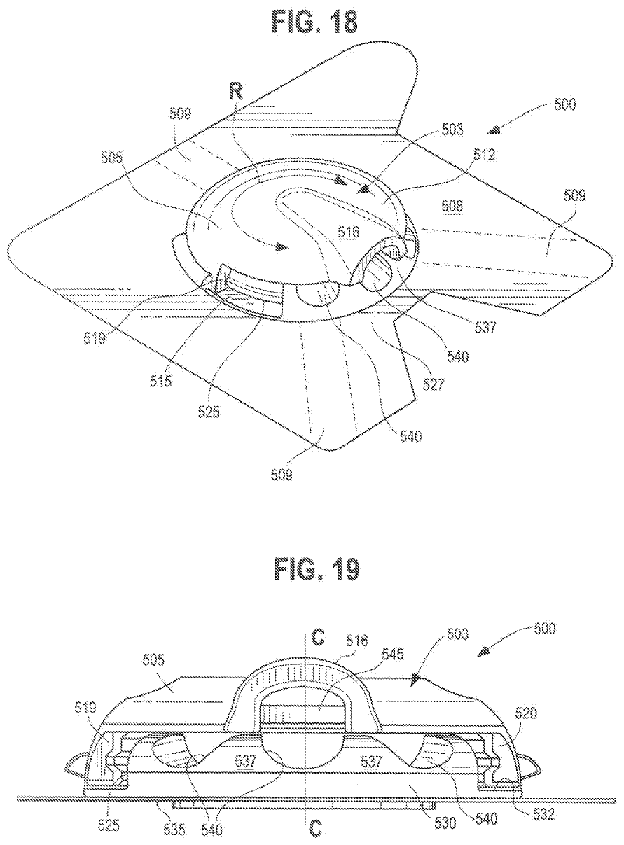

Referring to FIGS. 18 and 19, portions of a chest drainage system securing apparatus 500 having a reduced profile securement cap or guiding cover 503 operatively connected thereto and structured and arranged to reduce potential catch points are illustrated in accordance with an embodiment of the present invention. Similar in configuration and operation to the embodiment of FIGS. 1-3, the guiding cover 503 has a body 505 formed or molded, by way of example of a plastic, synthetic or other suitable material that is releasably attached to an adhesive pad 508 having a plurality of one way vents 509 formed therein. The guiding cover is structured and arranged to directionally align a chest tube 8 with an incision 24 (FIGS. 1-4A) and to position the hollow drainage conduit portion of a chest tube in conforming alignment with an outer surface of a patient's chest during the drainage procedure. The body 505 includes an upper or outer surface 512, a bottom or lower surface 515 and a radially outwardly extending guide channel or tunnel 516 formed therein structured and arranged to receive the chest tube when the guiding cover is aligned with and positioned on the adhesive pad, as, will be described in greater detail below. The guiding cover further includes a pair of securement clips 519, 520, extending axially downwardly from the body 505 in a direction substantially parallel with axis C-C of the securing apparatus. Each of the pair of securement clips is adapted align with and to releasably engage a channel or track 525 formed in and extending circumferentially around an edge 527 of a base 530 secured to the adhesive pad. In the embodiment shown, each of the clips is positioned circumferentially 180° apart; however, it is to be understood that the clips or other suitable fastening devices may be positioned on other portions of the guiding cover body 155 without departing from the scope of the present invention.

The base 530 includes an upper surface 532, a lower surface 535, a side or lip 537 located radially outwardly from and extending circumferentially along a segment of the edge 527 and axially upwardly from the upper surface 532 of the base. The side or lip 537 has a plurality of spaced-apart, radially outwardly extending guide channels 540 formed therein, each guide channel being adapted to receive a chest tube therein. As best shown in FIG. 9, the guiding cover 503 is adapted to be selectively rotatably positioned on the base 530 about axis C-C (as indicated by the arrow R) so that the guide channel or tunnel 516 is aligned with one of the plurality of guide channels 540 formed in the lip 537. Once in position on a patient, the guiding cover may be rotated such that the body 12 of a chest tube (as shown in FIG. 1) is directed down an outer surface of a patient's chest and to change the orientation of the chest tube inside the patient's pleural cavity. The tunnel and the selected one of the guide channels cooperate with one another to form a cylindrical port or aperture 545 (FIG. 10) adapted to receive and selectively adjustably secure a chest tube in a preselected position on a patient.

In another embodiment, a chest drainage system and securing apparatus 600 is depicted in FIGS. 20A and 20B and includes a chest tube 601 having a semi-rigid elongate body 602 which includes a patient end 603, a machine end 605 and a bend or curvilinear portion 606 that permits insertion of the patient end of the chest tube through an external incision made in a patient's skin 622 and chest wall 623 into the patient's chest cavity or pleural space. A continuous sidewall 604 extends between the machine end and the curvilinear portion, thereby forming a hollow drainage conduit adapted to drain air, blood, and fluids from the patient's chest cavity, as described above. A flutter valve (also known as a Heimlich-type one-way valve) 607 is operatively connected to the machine end 605 of the chest tube via a Luer lock connector 612. The flutter valve includes a body portion 606 enclosing a rubber sleeve 608 which only allows flow of air and fluids in a direction out of a patient's anatomical space 624 and not back into it. The chest drainage and securing apparatus 600 further includes an adhesive pad 615 having an aperture 617 formed therein for viewing visual placement of an incision and the chest tube.

The flutter or Heimlich valve includes a first or patient end 630 and a second or machine end 632. A female Luer connector 635 is operatively connected to the patient end 630 and adapted to receive a male Luer connector 837 attached to the machine end 505 of the chest tube. A Christmas tree connector or adaptor 639 is attached at a first end 640 thereof to the machine end 632 of the Heimlich valve and at a second end 642 to a second tube 644 positioned intermediate the Heimlich valve and a vacuum apparatus or other suitable drainage device as is known generally in the art.

The chest drainage system includes a securing and restraint apparatus designated generally at the numeral 650. The securing and restraint apparatus includes a tube clamp subassembly 655 which is operatively connected to adhesive pad 615 adapted to conform and be secured to the outer surface 622 of the patient's chest 623. The tube clamp assembly 655 is adapted to be releasably engaged with the chest tube 601 and is structured and arranged to securely grip the chest tube, whereby clinically significant movement of the chest tube with respect to a patient is prevented after it is inserted in a patient.

While only selected embodiments have been chosen to illustrate the present invention, it will be apparent to those skilled in the art from this disclosure that various changes and modifications can be made herein without departing from the scope of the invention as defined in the appended claim. The interactive restraint system herein disclosed may be used in connection with any form of catheter in addition to chest drainage systems and chest tubes illustrated herein. Furthermore, the foregoing descriptions of the embodiments according to the present invention are provided for illustration only, and not for the purpose of limiting the invention as defined by the appended claim and its equivalents.

What is claimed is:

1. A drainage system for removing air, blood, and other abnormal infectious or malignant fluids from an anatomical area in a human or, in veterinary applications, in an animal patient, the patient having body, the body including an outer surface having a contour, anatomical areas or spaces located in the body, a chest, a chest wall, a chest cavity or pleural space, a diaphragm, a torso, an outer surface skin, and lungs, the drainage system comprising:
a tubular drainage device having a semi-rigid elongate body, the semi-rigid elongate body including a patient end, a curvilinear portion operatively connected to the patient end, the curvilinear portion being adapted to permit insertion of the patient end of the tubular drainage device through an external incision or aperture formed in a patient's skin and in the outer surface of the patient's body and into an anatomical area of interest, a machine end, and a continuous sidewall extending between the machine end and the curvilinear portion, the continuous sidewall forming a hollow drainage portion or conduit adapted to conform and to be secured to the outer surface of the patient's body; and a drainage system securing apparatus or assembly, the drainage system securing assembly including a removable incision seal adhesive pad, an incision seal subassembly, and a tube clamp subassembly, the incision seal subassembly and the tube clamp subassembly being operatively connected to the removable incision seal adhesive pad.

2. The drainage system of claim 1 wherein the removable incision seal adhesive pad includes membrane body having an upper surface, a lower surface, a peripherally extending edge surface, a perforated seam or joint extending intermediate first and second opposing portions of the peripherally extending edge surface, the removable incision seal pad being separable into first and second adhesive pad segments along the perforated seam or joint.

3. The drainage system of claim 2 wherein the lower surface of the membrane body includes a hydrogel adhesive layer extending thereacross, the hydrogel adhesive layer being adapted to provide adhesive engagement with and conformation to the patient's outer body surface contour.

4. The drainage system of claim 3 further including a removable release liner extending across the hydrogel adhesive layer.

5. The drainage system of claim 4 wherein the incision seal subassembly is operatively connected to the first adhesive pad segment, and the tube clamp subassembly is operatively connected to the second adhesive pad segment.

6. The drainage system of claim 5 wherein the first adhesive pad segment includes an aperture extending between the upper surface and the lower surface circumferentially about and coaxially along an axis, the aperture being adapted to be removably placed over the external incision or aperture in a patient's skin and the outer surface of the patient's body.

7. The drainage system of claim 6 wherein the first adhesive pad segment includes a plurality of internal one-way vents or venting channels embedded or formed in the membrane body, each of the one way vents being in fluid communication via a first end thereof with the aperture and via a second end thereof with the peripherally extending edge surface of the membrane body, whereby air, blood, fluids, and disease and/or infection produced matter and by-products are removed from the anatomical area of interest in the patient but are prevented from flowing back into the anatomical area of interest.

8. The drainage system of claim 7 wherein the incision seal subassembly is operatively connected to the first adhesive pad segment, and the tube clamp subassembly is operatively connected to the second adhesive pad segment.

9. The drainage system of claim 8 wherein the incision seal subassembly includes a removable incision seal base frame adapted to fit securely into and in sealing engagement with the aperture formed in the first adhesive pad segment, a sealing member and an incision seal cap clamp removably positioned in an annular stepped surface formed in the removable incision seal base frame, an incision seal elastic membrane removably positioned intermediate the incision seal cap clamp and a removable incision seal cap, and a securement mechanism adapted to releasably secure the incision seal cap clamp, the incision seal elastic membrane, and the incision seal cap to one another.

10. The drainage system of claim 9 wherein the incision seal elastic membrane includes a body having an upper neck or curved nipple shaped body portion, a flat upper end having an aperture formed therein adapted to receive the patient end of the tubular drainage device, an incision seal plug cap removably positioned in the aperture formed in the flat upper end of the upper neck portion of the incision seal elastic membrane, and a split ring lock positioned circumferentially around the upper neck or curved nipple shaped body portion of the incision seal elastic membrane, the incision seal plug cap and the split ring lock cooperating with one another to seal the incision seal elastic membrane.

11. The drainage system of claim 10 wherein the securement mechanism adapted to releasably secure the incision seal cap clamp, the incision seal elastic membrane, and the incision seal cap to one another comprises a plurality of cap screws, each one of the plurality of cap screw being threadably received into a corresponding one of a plurality of cap screw inserts located in the removable incision seal cap.

12. The drainage system of claim 11 further including a tether having a first end operatively connected to the incision seal base frame and a second end operatively connected to the removable incision seal cap.

13. The drainage system of claim 12 wherein the tube clamp subassembly includes a tube clamp housing operatively connected to adhesive pad segment, a tube clamp base member releasably connected to the tube clamp housing, and a tube clamp top or cap member operatively connected to the tube clamp base member, the tube clamp top or cap being adapted to close in locking engagement with the tube clamp base member whereby the machine end of the tubular drainage device is releasably secured to the patient.

14. The drainage system of claim 13 wherein the tube clamp housing and tube clamp base member have aligned, oppositely disposed and spaced apart apertures or slots respectively formed therein, each aperture or slot being structured and arranged to releasably receive and secure the machine end of the tubular drainage device.

15. The drainage system of claim 14 wherein the tube clamp top or cap member includes an upper surface, a lower surface, and a flexible beam member operatively connected at a first end thereof to the lower surface, the flexible beam member having a second end adapted to be urged into locking engagement with a portion of the tubular drainage device.

16. The drainage system of claim 15 further including a rubberized or a silicone liner material coating on the tube clamp top member and the tube clamp base member to enhance frictional properties and corresponding retention strength thereof.

17. The drainage system of claim 16 further including a reduced profile securement cap operatively connected to the removable incision seal cap, the reduced profile securement cap being structured and arranged to reduce potential catch points of the system.

18. The drainage system of claim 16 further including a Heimlich-type one way valve operatively connected to the machine end of the tubular drainage device.

19. The drainage system of claim 18 wherein the Heimlich-type one way valve is operatively connected to the machine end of the tubular drainage device via a Luer lock connector.

20. The drainage system of claim 1 wherein the tubular drainage device comprises a chest tube.

21. A chest drainage system for removing air, blood, and other abnormal infectious or malignant fluids from a chest cavity or pleural space in a human or, in veterinary applications, in an animal patient, the patient having body, the body including an outer surface having a contour, anatomical areas or spaces located in the body, a chest, a chest wall, a chest cavity or pleural space, a diaphragm, a torso, an outer surface skin, and lungs, the drainage system comprising:
a chest tube having a semi-rigid elongate body, the semi-rigid elongate body including a patient end, a curvilinear portion operatively connected to the patient end, the curvilinear portion being adapted to permit insertion of the patient end of the chest tube through an external incision or aperture formed in a patient's skin and in the outer surface of the patient's body and into the patient's chest cavity or pleural space, a machine end, and a continuous sidewall extending between the machine end and the curvilinear portion, the continuous sidewall forming a hollow drainage portion or conduit adapted to conform and to be secured to the outer surface of the patient's chest cavity and torso; and
a drainage system securing apparatus or assembly, the drainage system securing assembly including a removable incision seal adhesive pad, an incision seal subassembly, and a tube clamp subassembly, the incision seal subassembly and the tube clamp subassembly being operatively connected to the removable incision seal adhesive pad.

22. The chest drainage system of claim 21 wherein the removable incision seal adhesive pad includes membrane body having an upper surface, a lower surface, a peripherally extending edge surface, a perforated seam or joint extending intermediate first and second opposing portions of the peripherally extending edge surface, the removable incision seal pad being separable into first and second adhesive pad segments along the perforated seam or joint.

23. The chest drainage system of claim 22 wherein the lower surface of the membrane body includes a hydrogel adhesive layer extending thereacross, the hydrogel adhesive layer being adapted to provide adhesive engagement with and conformation to a surface contour of the patient's chest and torso.

24. The chest drainage system of claim 23 further including a removable release liner extending across the hydrogel adhesive layer.

25. The chest drainage system of claim 24 wherein the incision seal subassembly is operatively connected to the first adhesive pad segment, and the tube clamp subassembly is operatively connected to the second adhesive pad segment.

26. The chest drainage system of claim 25 wherein the first adhesive pad segment includes an aperture extending between the upper surface and the lower surface circumferentially about and coaxially along an axis, the aperture being adapted to be removably placed over the external incision or aperture in a patient's skin and the outer surface of the patient's chest.

27. The chest drainage system of claim 26 wherein the first adhesive pad segment includes a plurality of internal one-way vents or venting channels embedded or formed in the membrane body, each of the one way vents being in fluid communication via a first end thereof with the aperture and via a second end thereof with the peripherally extending edge surface of the membrane body, whereby air, blood, fluids, and disease and/or infection produced matter and by-products are removed from the patient's chest cavity but are prevented from flowing back into the patient's chest cavity.

28. The chest drainage system of claim 27 wherein the incision seal subassembly is operatively connected to the first adhesive pad segment, and the tube clamp subassembly is operatively connected to the second adhesive pad segment.

29. The chest drainage system of claim 28 wherein the incision seal subassembly includes a removable incision seal base frame adapted to fit securely into and in sealing engagement with the aperture formed in the first adhesive pad segment, a sealing member and an incision seal cap clamp removably positioned in an annular stepped surface formed in the removable incision seal base frame, an incision seal elastic membrane removably positioned intermediate the incision seal cap clamp and a removable incision seal cap, and a securement mechanism adapted to releasably secure the incision seal cap clamp, the incision seal elastic membrane, and the incision seal cap to one another.

30. The chest drainage system of claim 29 wherein the incision seal elastic membrane includes a body having an upper neck or curved nipple shaped body portion, a fiat upper end having an aperture formed therein adapted to receive the patient end of the chest tube, an incision seal plug cap removably positioned in the aperture formed in the flat upper end of the upper neck portion of the incision seal elastic membrane, and a split ring lock positioned circumferentially around the upper neck or curved nipple shaped body portion of the incision seal elastic membrane, the incision seal plug cap and the split ring lock cooperating with one another to seal the incision seal elastic membrane.

31. The chest drainage system of claim 30 wherein the securement mechanism adapted to releasably secure the incision seal cap clamp, the incision seal elastic membrane, and the incision seal cap to one another comprises a plurality of cap screws, each one of the plurality of cap screw being threadably received into a corresponding one of a plurality of cap screw inserts located in the removable incision seal cap.

32. The chest drainage system of claim 31 further including a tether having a first end operatively connected to the incision seal base frame and a second end operatively connected to the removable incision seal cap.

33. The chest drainage system of claim 32 wherein the tube clamp subassembly includes a tube clamp housing operatively connected to adhesive pad segment, a tube clamp base member releasably connected to the tube clamp housing, and a tube clamp top or cap member operatively connected to the tube clamp base member, the tube clamp top or cap being adapted to close in locking engagement with the tube clamp base member whereby the machine end of the chest tube is releasably secured to the patient.

34. The chest drainage system of claim 33 wherein the tube clamp housing and tube clamp base member have aligned, oppositely disposed and spaced apart apertures or slots respectively formed therein, each aperture or slot being structured and arranged to releasably receive and secure the machine end of the chest tube.

35. The chest drainage system of claim 34 wherein the tube clamp top or cap member includes an upper surface, a lower surface, and a flexible beam member operatively connected at a first end thereof to the lower surface, the flexible beam member having a second end adapted to be urged into locking engagement with a portion of the chest tube.

36. The chest drainage system of claim 35 further including a rubberized or a silicone liner material coating on the tube clamp top member and the tube clamp base member to enhance frictional properties and corresponding retention strength thereof.

37. The chest drainage system of claim 36 further including a reduced profile securement cap operatively connected to the removable incision seal cap, the reduced profile securement cap being structured and arranged to reduce potential catch points of the system.

38. The chest drainage system of claim 36 further including a Heimlich-type one way valve operatively connected to the machine end of the chest tube.

39. The chest drainage system of claim 38 wherein the Heimlich-type one way valve is operatively connected to the machine end of the chest tube via a Luer lock connector.
